(19) United States
(12) Patent Application Publication
(10) Pub. No.: US 2010/0094267 A1
(43) Pub. Date: Apr. 15, 2010

(54) Confined Beam Laser Liposuction Handpiece

(76) Inventors: Jeff Ference, Charlottesville, VA (US); Daniel Geisert, Charlottesville, VA (US); Roger Hogue, Maple Grove, MN (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 12/630,333

(22) Filed: Dec. 3, 2009

Related U.S. Application Data
(63) Continuation-in-part of application No. PCT/US2008/083169, filed on Nov. 12, 2008.
(60) Provisional application No. 60/987,256, filed on Nov. 12, 2007.
(60) Provisional application No. 61/058,021, filed on Jun. 2, 2008.
(60) Provisional application No. 61/100,047, filed on Sep. 25, 2008.

(51) Int. Cl. A61B 18/18

(52) U.S. Cl. 606/15

(57) ABSTRACT

A cannula for a liposuction device includes a laser energy source for liquefying, emulsifying or softening fatty tissue at the cutting windows inside the distal tip end of the cannula. The energy source provides laser energy through an optical fiber positioned within said interior region of the cannula and extending though the cannula and having an end located at the distal tip end of the cannula adjacent the cutting windows. Fat is emulsified, liquefied or softened by the energy emitted into tissue at the cutting windows of the cannula and is removed by aspiration. In a confined laser beam power assisted embodiment, the openings in the cannula shear or cut neighboring fat tissue and the radiant energy at the cutting windows softens and/or emulsifies the tissue.

### CROSS-REFERENCE TO RELATED APPLICATIONS

This application is a continuation-in-part (CIP) application of International Application No. PCT/US2008/083169 filed Nov. 12, 2008, which claims priority to U.S. Provisional Patent Application 60/987,256 filed Nov. 12, 2007, U.S. Provisional Application 61/058,021 filed Jun. 2, 2008, and U.S. Provisional Application 61/100,047 filed Sep. 25, 2008, and the complete contents of these applications is herein incorporated by reference.

### BACKGROUND OF THE INVENTION

1. Field of the Invention

The present invention generally relates to surgical instruments used in liposuction surgical operations and, more particularly, to a novel confined beam laser liposuction (CBLL) handpiece which provides improved performance in liposuction surgical operations.

2. Background Description

Liposuction is a well known surgical procedure for surgically removing fat tissue from selected portions of a patient's body. Current practice is to make an incision and then insert a cannula in the space occupied by fat tissue. The cannula is then moved in such a manner as to mechanically break up the fat tissue. While moving the cannula, pieces of the fat tissue are aspirated from the space through the cannula by vacuum pressure from a syringe or pump. This technique requires significant effort on the part of the surgeon in terms of both the physical effort required to move the cannula back and forth, and the effort required to control the direction of movement of the cannula in order for fat tissue to be withdrawn only from specific areas of the patient's body.

U.S. Pat. No. 4,886,491 to Parisi et al. discloses a surgical instrument which utilizes an ultrasonic probe to break up fat tissue. U.S. Pat. No. 5,295,955 to Rosen discloses a surgical instrument which employs microwave energy to soften fat tissue. These approaches may produce a lumpy surface upon completion of the surgery.

Swartz discloses, in U.S. Pat. Nos. 4,735,605, 4,775,365, and 4,932,935, power assisted liposuction tools which include an external sheath which houses a rotary driven auger type element. Fat tissue is selectively sheared at an opening in the external sheath by the auger element pulling tissue within the opening and shearing it off at the opening. In one of the designs, Swartz contemplates oscillating the direction of rotation of the auger element. U.S. Pat. No. 4,815,462 to Clark discloses a lipectomy tool which has an inner cannula with a knife edge opening which rotates within an outer cannula. In Clark, fat tissue is drawn by suction into an opening the outer cannula, and is then sheared off by the knife edge of the inner cannula and aspirated to a collection vessel.

A disadvantage with each of these Swartz and Clark designs is that they may tear the tissue. This can be problematic when working in confined spaces near blood vessels and the like. U.S. Pat. No. 5,112,302 to Cucin discloses a powered liposuction hand tool that moves a cannula back and forth in a reciprocating manner. Back and forth movement is akin to the movements made by surgeons, and is therefore a marked improvement over the rotary designs of Swartz and Clark. However, the Cucin design requires the cannula and reciprocating mechanism to move within a portion of the hand held base unit.

U.S. Pat. No. 5,352,194 to Greco et al. describes an automated liposuction device with reciprocating cannula movement that is akin to Cucin's; however, this device relies on a pneumatic cylinder drive system, with multiple sensors, and a computer controller to adjust and regulate the cannula movement. Overall, the Greco system is complex and may be subject to a variety of drive control problems, as well as high costs for various elements. In addition, the Greco system is designed to provide cannula stroke lengths which are in excess of 1 cm, which may not be ideal in a number of different circumstances.

U.S. Pat. No. 5,348,535 to Cucin discloses another power assisted liposuction instrument. This instrument utilizes movement, of an internal sleeve within an external sleeve to shear off fat tissue pulled within an opening in the external sleeve. The design is complex in that it requires multiple sleeves, and the reciprocating movement causes periodic changes in the aspiration aperture.

U.S. Pat. No. 4,536,180 to Johnson discloses a surgical system for suction lipolysis which employs an internal or external air conduit which directs airflow at or near the cutting tip of the cannula to enhance fat tissue clearance during aspiration through the cannula.

U.S. Pat. No. 5,013,300 to Williams discloses suction lipectomy tool which allows suction control via the surgeons thumb covering and uncovering vent holes in the lipectomy tool housing.

U.S. Pat. No. 6,464,694 to Massengill discloses a liposuction cannula which has a source of aqueous solution, a laser source, and a suction source. Laser energy is emitted at the distal end of the cannula and simultaneously one or more jet streams of water or aqueous solution are emitted into the same area. As a result of the laser bombardment, the water molecules supplied by the jet stream are stated to be “hyperkinetized”. When released violently from the tip of the cannula, the water molecules disrupt the wall of the adipocyte cells and release the liquefied fat which is aspirated by the cannula.

U.S. Pat. No. 6,206,873 to Paolini et al. describes a device and method for removing subcutaneous adipose layers with a laser source, optical fiber and a hollow needle for guiding the fiber. The laser beam is stated to be generated with an intensity and wavelength for liquefying and maintaining liquid the adipose cells so that the membranes of adipose cells are disrupted without substantially damaging collagen in the adipose layers. The liquid material may be suctioned by means of a vacuum pump. However, the suction line is not shown to be incorporated into the device.

U.S. Pat. No. 6,902,559 to Taufig describes a liposuction device for removing subcutaneous fatty tissue. The Taufig device comprises a suction cannula including openings for sucking fatty tissue as well as an injection line with an injection opening disposed at the front injection line end for injecting a working fluid. For loosening the tissue, an ultrasonic generator is arranged near the injection opening of the suction cannula. Additionally, or alternatively, a laser may be provided at the suction cannula for heating and detaching the fatty tissue.

U.S. Pat. No. 5,642,370 to Mitchell et al. describes a medical laser device for ablating and emulsifying biological material. The laser, including erbium:YAG (yttrium aluminum garnet) gain medium, is stated to be optimized to generate a pulsed output of preferably at least 100 Hertz which is delivered to the target tissue via an optical fiber. A suction source is provided to aspirate the tissue as it is being ablated. This laser system provides accurate, ablation with minimal damage to surrounding tissue. In addition to ophthalmic and urinary organ procedures, the targeted removal of cancerous tissue and/or tumors and procedures, the erbium laser can be used for removal of fatty tissue.

U.S. Pat. No. 6,176,854 to Cone discloses a method of percutaneous and subcutaneous laser treatment of the tissue of a patient. The light energy is introduced into the proximal end portion of the optical fiber, passes through a handpiece and is emitted from the bare distal tip of the optical fiber. The tip of the optical fiber is passed through the skin and advanced through the tissue subcutaneously to a desired treatment area. Laser energy can be emitted at different levels during any or all of the skin penetration, advancement, tissue treatment and withdrawal phases.

MicroAire Surgical Instruments has been making a product known as the “PAL” for power assisted liposuction. The product represents a substantial improvement over previous liposuction handpieces, and implements features described in U.S. Pat. Nos. 5,911,700, 6,139,518, 6,258,054, and 6,817,996.

### SUMMARY OF THE INVENTION

It is therefore an object of the present invention to provide a novel confined beam laser liposuction (CBLL) handpiece which provides improved performance in liposuction surgical operations.

According to the invention, there is provided an improved, power-assisted, reciprocating liposuction tool which includes a source of laser energy confined within the cannula tip region which emits laser light at power levels sufficient to soften or melt adipose tissue which is aspirated by the liposuction tool. In a particular embodiment, the source of laser light is either continuously or periodically energized. In addition, the laser is preferably introduced though the handpiece into the cannula using a Y connector that allows an optical fiber to pass through the aspiration tubing and handpiece and extend into the hollow cannula. Further, the interior of the cannula, or at least a portion of the interior of the cannula near the end of the optical fiber from which the laser energy emanates is coated with a reflective coating to reflect energy inside the cannula tip and to maintain the external surface of the cannula at a low temperature (i.e., a temperature that is lower than if the energy projected on the inside of the cannula were permitted to heat the cannula).

According to an embodiment of the invention, a laser source is combined with a power-assisted liposuction tool and confined within the cannula tip near the cutting window(s) (one or more openings in the cannula) used for shearing fat tissue. The cannula is provided with an optical fiber which carries the laser light and extends from the laser source through the handpiece and the length of the cannula until just prior to its distal end at the opening used for shearing fat tissue. The optical fiber can be introduced through the aspiration tubing via a Y connector and into the handpiece and cannula. Laser energy delivered by the optical fiber to the targeted fatty tissues which are present within the openings used for shearing fat tissue should be of sufficient energy strength to be able to emulsify, soften, or liquefy the fat at the treatment site.

According to yet another embodiment of the invention, the powered surgical handpiece includes a reciprocating member to which a cannula is connected. The handpiece drives the cannula back and forth under the control of a drive mechanism that preferably provides for variable speeds of reciprocation. The handpiece can employ any type of drive mechanism, including for example electric or pneumatic variable speed drive. In the preferred embodiment, cannulas are connected external to the hand piece by a connector which secures the cannula to a reciprocating member. The connector can either be integral with the cannula, integral with the reciprocating member or constitute a piece which is separate from and connectable to each of the reciprocating member and the cannula. In the most preferred embodiment, the connector is separate from the reciprocating member, and is designed to quickly connect to and disconnect from the reciprocating member by a pushbutton fitting or similar device. In the preferred configuration, the connector spaces the cannula radially from the axis of the reciprocating member such that the when the cannula is installed, it moves in a reciprocating motion along an axis that is parallel to the axis of the reciprocating member. The offset thus created allows the cannula to be positioned in alignment with a vacuum hose or other vacuum mechanism, such that fat tissue will be freely aspirated through the cannula into the vacuum tube. In the most preferred configuration, the vacuum hose fits directly onto the end of the cannula and an optical fiber carrying laser energy fits through the vacuum hose and extends into the handpiece and up towards the end of the cannula such that it can direct laser energy towards fat tissue that is sheared off or is in the process of being sheared off at the openings in the end of the cannula.

In one embodiment, the power-assisted liposuction tool includes a series of one or more holes disposed on the outside wall and distal end of the cannula. The cannula holes are used to aspirate into the vacuum hose the fat liquefied by laser energy, and therefore to remove liquefied fat from the treatment site. Also, it is preferred that the forward and rearward stroke length of the cannula can be set to be equal to or greater than the size of the cutting window or windows in the cannula.

The power assisted liposuction tool preferably supplements the movements currently used in liposuction procedures. That is, it has been found that the reciprocal movements of the cannula, which may be 0.1 to 6 mm in length, tend to make it significantly easier for the surgeon to move the cannula back and forth in the same manner as is done with a non-power assisted liposuction tool. The precise reason for the reduction in force required is not known but may be related to enhanced fat bursting attributed to the head of the cannula and window sections being moved into and across the fat cells in a repetitive motion while the cannula is being manually moved forward and rearward by the surgeon. Preferably, the power assisted liposuction handpiece will allow regulation of the suction pressure applied and/or the stroke length of the cannula (i.e., the distance the cannula tip travels from its fully extended to fully retracted positions in one reciprocal motion) and/or the degree of energy emitted by the energy source within the confined cannula tip. In this way, the tool can be used for excising different types of tissue and for working on different types of body fat. For example, it will be understood by one of ordinary skill in the art that the requirements of a liposuction tool in the neck region are different from those in the abdomen and/or legs. The liposuction handpiece of the present invention can be designed to allow for the interchange of cannulas using the same handpiece, the regulation of reciprocation speed, the regulation of suction, and the regulation of stroke length, thereby allowing the same tool to be used in a variety of applications and to meet the needs and desires of several different specialists.

### BRIEF DESCRIPTION OF THE DRAWINGS

The foregoing and other objects, aspects and advantages will be better understood from the following detailed description of a preferred embodiment of the invention with reference to the drawings, in which:

FIGS. 1A and 1B are side views of an exemplary power assisted, liposuction/lipoinjection tool according to the present invention respectively showing a cannula disconnected and connected to a reciprocating member of the handpiece;

FIG. 2 is a bottom plan view of the exemplary power assisted liposuction/lipoinjection tool showing hose clamping slots formed in the handle region;

FIG. 3 is a top view of a cannula connector;

FIG. 4 is a cross-sectional view of a cannula connector;

FIG. 5 is a cut-away cross-sectional view of a portion of a connector affixed to a reciprocating member of the handpiece, with a vacuum hose attached to the cannula end;

FIG. 6 is a side view of a connector which is integral with a reciprocating member and which is selectively connectable to and disengageable from disposable or re-usable cannulas;

FIG. 7 is an end view of a connector which can selectively connect different cannulas;

FIGS. 8A to 8D are plan views of several different cannula tips showing a variety of different window configurations;

FIG. 9 is a schematic of the exemplary liposuction/lipoinjection equipment showing collection of fat tissue in a filter, and suction control;

FIG. 10 is a schematic cross-sectional view of a cannula with an internal fluid or gas delivery tube;

FIGS. 11A and 11B are side views of exemplary alternative power-assisted liposuction handpieces, each having a branched cannula;

FIG. 12 is an image of an exemplary power-assisted laser liposuction handpiece having a cannula with a laser added to the tip;

FIG. 13 is a cut-away cross-sectional view of a cannula with added laser;

FIG. 14 is a cut-away cross-sectional view of a cannula with the end of an optical fiber which emanates radiant energy near the cannula cutting windows;

FIG. 15 is a schematic drawing showing the laser connected to an optical fiber which is positioned within the aspiration tubing and extends through the power assisted liposuction handpiece and to the cutting windows near the cannula tip; and

FIG. 16 is an isometric view of a powered liposuction handpiece with confined beam laser liposuction (CBLL) capability in the hand of a surgeon.

### DETAILED DESCRIPTION OF A PREFERRED EMBODIMENT OF THE INVENTION

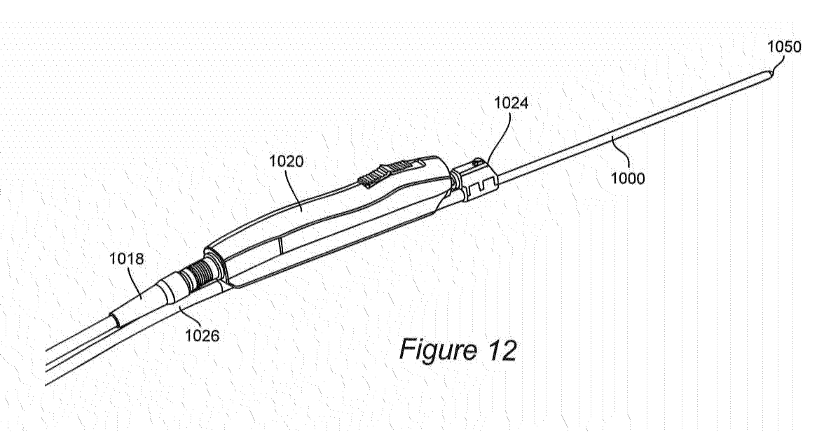

The preferred embodiment of the invention is practiced with a power assisted liposuction tool such as the “PAL®” product sold by MicroAire Surgical Instruments of Charlottesville, Va. In particular, the energy application devices as best shown in FIGS. 12-15 of this patent application may be incorporated into a power assisted liposuction tool as described in U.S. Pat. Nos. 5,911,700, 6,139,518, 6,258,054, and 6,817,996, each of which are herein incorporated by reference. However, it should also be understood that the energy application devices and configurations may be employed with other power assisted liposuction tools in the practice of this invention. That is, any liposuction cannula which reciprocates or vibrates, which includes an aspiration mechanism, can benefit from having an energy source which delivers electromagnetic radiation or other energy from its tip area to emulsify, soften or liquefy fat tissue inside the tip region of the cannula. Thus, for exemplary purposes only, the invention is described in conjunction with the features of a power assisted liposuction tool as described in U.S. Pat. Nos. 5,911,700, 6,139,518, 6,258,054, and 6,817,996.

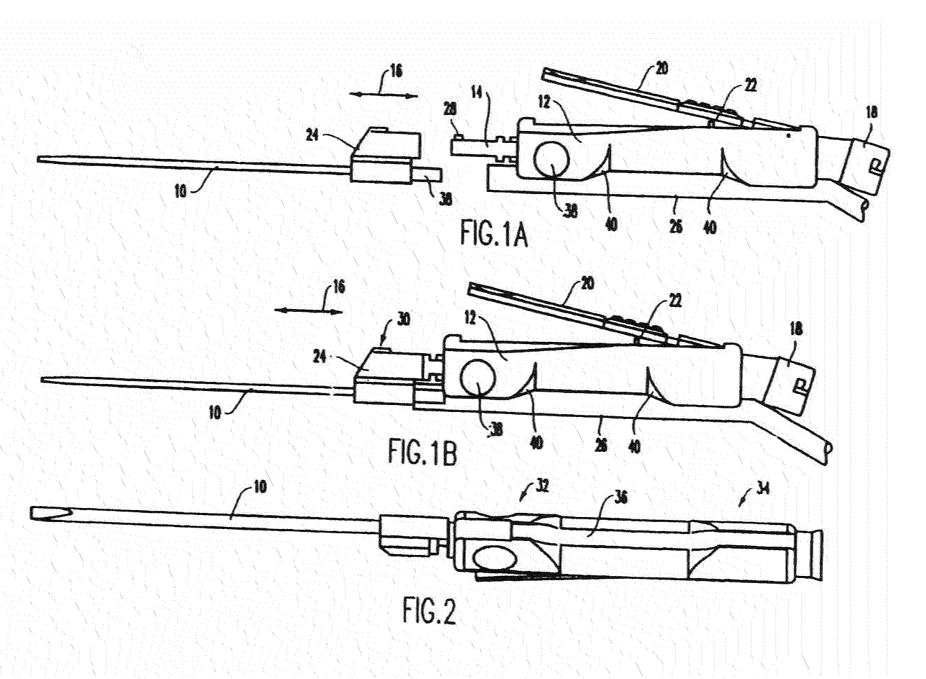

Referring now to the drawings, and more particularly to FIGS. 1A and 1B, there is shown the preferred embodiment of an exemplary power assisted liposuction/lipoinjection handpiece. A cannula 10 is selectively connectable and disconnectable from a handle 12. The handle 12 includes a reciprocating member 14 which moves back and forth, as indicated by double headed arrow 16, in a reciprocating motion. In the preferred embodiment, the handle 12 includes a pneumatic drive assembly (not shown) and is connectable to a compressed air source by connector 18. An example of a suitable handle with internal pneumatic drive could be the MicroAire® 1400-100. However, it should be understood that any drive mechanism, including electrical, magnetic, etc., can be used to move the cannula 10 in a reciprocating motion 16.

The speed of reciprocation is preferably variable under the control of a lever 20 actuated button or switch 22, whereby complete depression of the lever 20 accelerates the reciprocation to its maximum speed, and partial depression of the lever 20 accelerates the reciprocation to speeds which are less than maximum speed. This enables the surgeon to adjust the speed as conditions require. However, it will be apparent to those skilled in the art that the liposuction tool could employ a simple on/off switch with a preset speed of reciprocation, or a series of pre-set speed buttons which allow the surgeon to selectively alter the reciprocation speed to any pre-established level. The optimum speed of reciprocation 16 may vary for different liposuction operations and/or from patient to patient. It is expected that for most liposuction operations, a maximum speed ranging from 10-100,000 cycles/minute will be suitable. While not shown, the handle 12 could be equipped with sensors and protection circuits which sense the speed of reciprocation 16, and prevent the speed from exceeding a pre-set level, where the pre-set level could be established to protect either the patient or drive mechanism inside the handle 12.

While FIGS. 1A and 1B show a “wand” style handle 12, it will be understood by those of skill in the art that the configuration of the handle can vary widely to meet the needs or desires of the surgeon. Thus, the handle 12 could take the form of a pistol grip or other configuration, and the lever 22 could take the form of a trigger or other suitable mechanism. In the preferred embodiment, the stoke length, which is defined as the difference between the furthest point to which the cannula 10 extends and the shortest point cannula 10 extends in one reciprocating movement 16, will preferably be greater than 0.1 mm and less than 1 cm. The preferred range in most applications will be 1-60 mm, and the most preferred is 1-3 mm. While the reciprocating motion 16 itself will allow for breaking up fat particles and aspiration of fat, it is expected that the surgeon will still move the cannula 10 back and forth, or in any other direction, during the liposuction procedure; thereby removing fat from areas he or she deems most appropriate. The reciprocating motion 16 enhances the surgeon's ability to move the cannula 10 after it has been inserted into the patient. When the cannula 10 is being reciprocated by a powered mechanism, particularly for short lengths of less than 1 cm, it is physically easier for the surgeon to move the cannula 10 through material to be aspirated. In this sense, the liposuction tool supplements the motions and procedures currently used by surgeons by making them easier and less tiring to perform. However, for certain procedures, the reciprocating movement 16 might serve as a complete replacement for back and forth movements made by the surgeon.

While not specifically shown in FIGS. 1A and 1B, a switch or dial or other suitable control structure may be associated with the handle 12 to allow the surgeon to change the stroke length for the cannula to meet his or her requirements for different applications. This control structure would then limit the movement of reciprocating member 14 to desired distance.

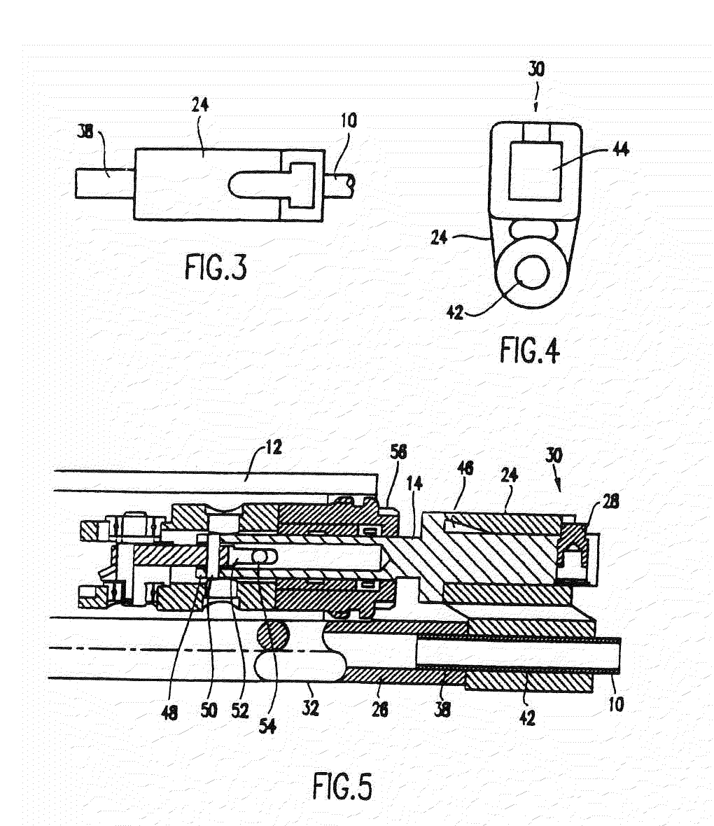

In one embodiment, a connector 24 or other suitable device, secures the cannula 10 to the reciprocating member 14 and to a vacuum hose 26 or other suitable source of vacuum pressure. Preferably, a push-button 28 or other selectively actuatable member on the reciprocating member 14 will be used to install and lock the connector 24 to the reciprocating member 14, such that the cannula 10 will be safely retained on the handle 12 during liposuction. Push-button 28 is depressed as it enters a bore passage in the connector 24, and when the connector is correctly installed the push-button returns to the upright position and is locked within a locking region 30 of the connector 24. To remove the cannula 10, the surgeon simply depresses the push-button 28, and slides the connector 24 off the reciprocating member 14. The connector 24 and its installation on the reciprocating member are discussed in more detail below in conjunction with FIGS. 3 to 5. It should be understood that other locking mechanisms besides push-buttons 28 could be used within the practice of this invention, including for example latch mechanisms, pin mechanisms, and the like.

FIG. 2 shows that in one embodiment, the vacuum hose 26 is secured to the handle 12 via hose clamping slots 32 and 34 formed on the base of the handle 24. The hose clamping slots 32 and 34 are open at the base so that the vacuum hose can be press-fit in place on the bottom of the handle 24 along region 36. This allows the surgeon's hand to comfortably hold the handle 12 without becoming entangled with the hose 26, and assures that the hose 26 remains firmly in place during operation of the liposuction/lipoinjection equipment. To enhance the ergonomics of the handle 12, cut-out spheres 39, and contours 40 can be provided.

To allow aspiration of fat tissue from the cannula, the vacuum hose 26 is fitted onto hose engaging member 38 at the rear of cannula 10 (or, alternatively a projection on the connector 24). The hose engaging member 38 preferably takes the form of a hollow cylinder or a polygonal conduit which is wider in cross-section than the portion of the cannula 10 which is extended into the patient; however, it may be desirable to simply have the hose engaging member 38 simply be the end of the cannula 10. All that is required is that the hose 26 fit onto the hose engaging member 38 and be securely held thereto.

It should be understood that the hose engaging member 38 can either be part of the connector 24 or be part of the cannula 10. In the embodiment where the hose engaging member 38 is part of the connector 24, a passage (not shown) through the connector 24 allows vacuum communication between the cannula 10 and the hose 26. However, in a particular embodiment, the cannula 10 is directly connectable to the hose 26. In the configuration shown in FIGS. 1A and 1B, the cannula 10 extends through the connector 24 and its base would be the hose engaging Member 38, and the thickness of the base would, if desired, be widened or made polygonal so that it fits snugly within the internal diameter of the hose.

The vacuum hose 26 will preferably be optically clear, thus allowing the surgeon to determine if the hose 26 is clogged with fat tissue aspirated from the patient's body through the cannula. By monitoring the vacuum pressure and hose line, the surgeon can determine when corrective measures need to be taken during liposuction. Polyvinylchloride is an example of a suitable material for the hose 26. The chief requirements for the hose 26 is that it be flexible enough that it be able to be press-fit within and retained by the hose clamping slots 32 and 34, it be sufficiently “stretchable”, “pliable” or the like, that it can stretch with reciprocating movements 16 of the cannula without being released from the hose engaging member 38, and have a sufficient internal diameter (not shown) to allow fat tissue and fluids aspirated from the patient's body to flow to a collection vessel or filter. As will be discussed in more detail below in connection with FIG. 15, in a preferred embodiment an optical fiber which transports laser energy to the confined end of the cannula where the cutting windows are located preferably fits within the aspiration hose 26 and extends into the cannula 10.

The design shown in FIGS. 1A and 1B shows an embodiment of this invention where the cannula 10 is offset radially from the axis of the reciprocating member 14 such that it is in direct alignment with the vacuum hose 26. Thus, the cannula 10 reciprocates along an axis which is parallel to the reciprocating member 14, but which is in alignment with the section of the vacuum hose 10 affixed to the handle 12. Alignment of the cannula 10 and vacuum hose 26 eliminates bent regions and, thereby enhances the ability of vacuum pressure to aspirate fat tissue through the cannula 10 into the vacuum hose 26. Furthermore, the alignment makes it easier for the vacuum hose to remain affixed during reciprocation of the cannula 10, as well as making it simpler to affix the connector 24 to the reciprocating member 14 and hose 26. While the design in FIGS. 1A and 1B provides for neat storage of the hose 26, in some applications it may be desired to have the hose 26 more directly clamped to the cannula (e.g., by a hose clamp or other suitable device), and be freely moveable therewith. In this embodiment, the hose 26 would simply not be stowed under the handle 12 as shown, or, if the invention took the form of a pistol grip design, the hose would simply project off to one side or be oriented in any other convenient manner which preferably does not interfere with the surgical operations being performed.

Having the cannula 10 disconnectable from the reciprocating member 14 provides advantages in terms of cleaning and or disposal: however, it should be understood that more permanent connections can be made. In some applications the cannula might be directly connected to the handle 12, such as by a connection of the cannula 10 directly to a reciprocating drive mechanism, rather than to an intermediate reciprocating member 14.

FIGS. 1A and 1B show an embodiment of the invention where the cannula 10 and connector 24 are be more or less permanently joined together. That is, they are integral such that the cannula 10/connector 24 combination form a self-contained unit which can be selectively installed on the handle 12. In this way, the cannula 10/connector 24 can be sterilized together, and packaged in tubes or sterile packages for later shipment and use. Thus, when required by the surgeon, the package will be opened in the operating room and cannula 10 will be connected to the handle 12 in one step. The cannula 10 and connector 24 can be made from the same or different materials. In this embodiment the cannula 10 is a hollow metal tube and the connector is made from plastic. The cannula 10 and connector 24 can be permanently bonded together by an adhesive to create an integral structure, or simply be connected by a friction fit.

FIGS. 3 to 5 show additional details where the cannula 10 is affixed to a connector 24. In FIG. 3, the hose engaging member 38 at the rear end of the cannula 10 is shown as an enlarged conduit which is either integral with or affixed to the cannula 10. Conversely, in FIG. 5, the rear end of the cannula 10 is not enlarged and the vacuum hose 24 is affixed directly to the base of the cannula 10. In either case, the cannula 10 extends through a cylindrical bore 42 in the connector 24. The vacuum hose 26 is held on the handle 12 by the hose clamping slot 32 shown in partial cross-section, and the inner diameter of the hose 26 is in alignment with the inner diameter of the cannula 10 such that fat tissue broken or sliced off from a patient, or which is liquefied using laser or other energy output as described in more detail below, moves through the cannula 10 into the hose 26 and to a collection vessel. As explained above, the offset provided by the connector 24 assures proper alignment of the hose 25 and cannula 10.

The vacuum hose 26 under the handle 12, in one embodiment, does not move in conjunction with the reciprocating motion of the cannula 10 caused by the reciprocating member 14. Rather, the hose 26 could elongate and contract with each reciprocal stroke of the cannula. Alternatively, the cannula 10 could move freely within the inner diameter of the vacuum hose 26. In this case, the stroke length for the cannula 10 would need to be less than the length of the hose engaging end of the cannula 38 protruding from the connector 24, such that the hose remains connected to the cannula at all times. As a further alternative, as discussed above, the hose 24 could be clamped to the hose engaging member 38 of the cannula 10 and could be freely movable therewith; however, this alternative does not take advantage of the neat and clean hose storage feature of this invention.

The connector 24, in one embodiment, includes a square bore 44 for connecting with the reciprocating member 14. Making the reciprocating member 14 polygonal in shape assists in preventing the connector 24 from rotating axially about the reciprocating member 14 during high speed reciprocation. To affix the connector 24 on the reciprocating member 14, the reciprocating member 14 is inserted into square bore 44. An incline 46 formed in the connector 24 depresses the pushbutton 28. However, once the pushbutton 28 reaches locking region 30, it moves upward, via a spring mechanism or by other suitable means, and locks the connector 24 onto the reciprocating member 14.

If desired, the reciprocating member 14 could be removed from the handle 12 to allow connecting other tools (e.g., saw blades, drill bits, etc.) to the same handle 12. As indicated above, a suitable powered handle could be the MicroAire.®. 1400-000 which is used for driving reciprocating saw blades. Thus, if multi-tool functionality is desired, the reciprocating member 14 can be equipped with a drive connecting end 48 that fits on a pin connector 50. The reciprocating member 14 may also have a guide slot 52 which slides on pin guide 54 during reciprocating movements. The reciprocating member 14 would be disconnected by removing securing ring from the front of the handle 12, and then disconnecting the drive connecting end 48 from the pin connector 50. This feature may also be used to connect larger and smaller reciprocating members, or reciprocating members having different shapes to the same handle 12.

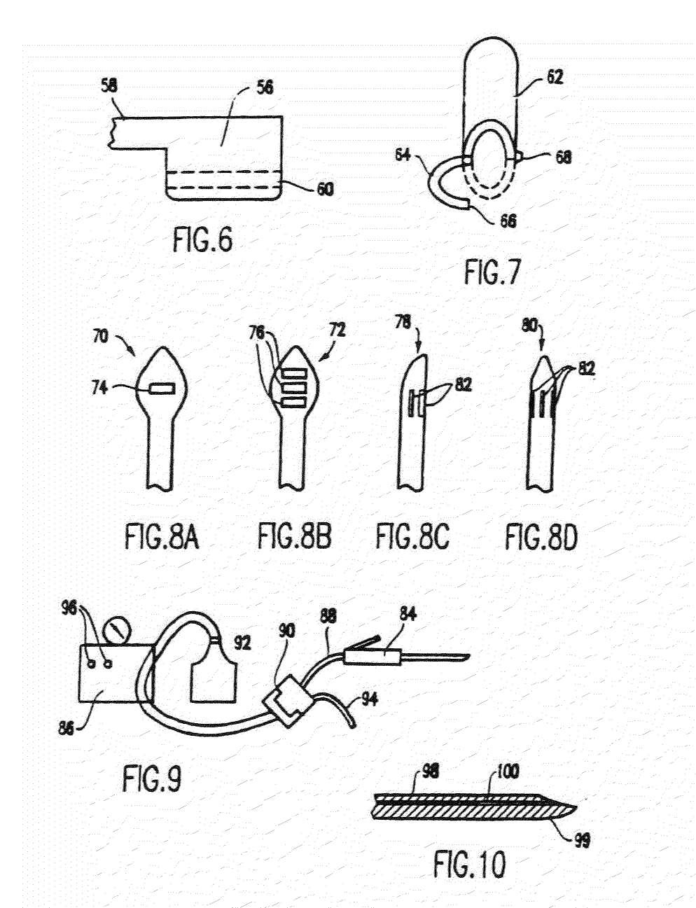

With reference back to FIGS. 1A and 1B, in some applications the cannula 10 could be disconnectable from the connector 24. To aid installation and reduce connecting operations needed by the surgeon, the connector 24 could be formed as an integral part of the reciprocating member. FIGS. 6 and 7 show alternative designs for a connector where the cannula can be disconnected. By allowing the cannula to be disconnected and connected as desired, the cannula configuration can be very simple (i.e., a hollow tube, preferably made of metal, with one or more cutting windows).

FIG. 6 shows a connector 56 which is integral with a reciprocating portion 58 which is fitted to a reciprocating drive mechanism (not shown). The connector has a bore hole 60 which extends through the length of the connector 56. Cannulas (not shown) can be connected and/or disconnected from the connector 56 by inserting them through the bore hole 60. A friction engagement, which can be supplemented with glue or other adhesives, holds the cannula within the bore hole 60. While connector 56 is shown as being integral with reciprocating portion 58, it should be understood that the same connector 56, which allows for selective attachment and/or disengagement of desired cannulas thereto, could be attachable to a separate reciprocating member 14, as is shown in FIGS. 1A and 1B.

FIG. 7 shows an alternative embodiment where a connector 62 includes a cannula locking portion 64 which rotates between an open position and a closed position (shown in dashed lines). A cannula (not shown) is inserted in the space between the connector 62 and locking portion 64, and is secured to the connector 62 by shutting the locking portion 64 and securing its free end 66 by a lock 68 or other securing member. To disengage the cannula, the lock 68 is released, and the locking portion 64 of the connector is pivoted away from the connector 62 body.

FIGS. 8A to 8D show several examples of cannula tips. It should be understood that any type of cannula tip can be used in the practice of the present invention. As will be discussed in more detail below, a laser diode or fiber optic or waveguide can extend to just inside the tip at the cutting windows to project laser energy into the surrounding adipose tissue at the cutting windows (or opening(s)) to soften, liquefy or emulsify fat tissue near the cutting windows of the cannula.

FIGS. 8A and 8B show cannulas 70 and 72 with spatula shaped heads. These types of cannulas are preferred in facial surgery and other types of liposuction where there is a need to separate fat from skin and muscle tissue and where space requirements are restricted. The spatula shaped head aids in separating the tissues. The face of the spatula shaped head can have a single cutting window 74 or a plurality of cutting windows 76. The shape of the cutting window 74 or 76 can vary to suit the needs of the surgeon. While oval windows are commonly employed, it has been determined that square or rectangular windows 74 and 76 are preferred for spatula shaped heads since they tend to allow for more accurate shaving and sculpting of tissue. In facial surgery, in addition to allowing for aspirating fat tissue from the patient's body, the cutting window 74 or 76 tends to be used to cut tissue from the patient's body during each reciprocal motion. Therefore, it is preferred to have the stroke length of the cannula be equal to or larger than the longitudinal distance from the bottom of the cutting window to the top of the cutting window. In this way, each reciprocating stroke of the cannula 70 or 72 will slice off a piece of fat tissue for subsequent aspiration. By keeping the stroke length small (e.g., 1-3 mm) and the longitudinal length of the window 74 or 76 small (e.g., less than or equal to 1-3 mm), fat particles of a small size are excised, and these fat particles are less likely to clog the vacuum hose or cannula. Further, emulsifying or melting the fat particles at the cutting window using radiant energy or other energy helps assure that there will be reduced clogging in the vacuum hose or cannula.

FIGS. 8C and 8D show cannulas 78 and 80 which are commonly used in full body or abdomen liposuction. FIG. 8C shows a blunt end cannula 78, and FIG. 8D shows a bullet end cannula 80. Each of these cannulas have one or a plurality of windows 82, which are typically oval shaped, around the periphery of the cannula near the tip of the cannula 78 or 80. In this type of liposuction, the reciprocating movement of the cannula 78 or 80, as well as the forward and backward movements of the entire handpiece made by the surgeon, tends to break up fat particles. The fluids and particles which are released from these motions, or which are liquefied or emulsified using radiant energy delivered from the tip region, are simply aspirated through the windows 82 in the cannula 78 or 80. In these applications, slicing by the windows 82 may or may not occur.

FIG. 9 shows a reciprocating liposuction tool 84 connected to a pump 86 or other vacuum pressure producing device. Fat aspirated through the cannula into the vacuum hose 88 is collected in a filter 90. The filter 90 should have openings which are large enough to allow fluids such as blood, plasma, etc. to pass through, but be small enough to allow larger fat particles to be collected. Preferably the filter 90 can be placed directly in line with the hose 88 or be integral with the hose 88. Fluids including blood pass through the filter 90 and are collected in collection vessel 92.

Collected fat tissue is typically used for lipoinjection procedures. Thus, by collecting the fat from a liposuction operation in a filter 90, the collected fat tissue can be more easily washed and then re-used in a lipoinjection procedure. In order to wash the collected fat, one would only need to remove the filter 90 and run wash or lavage fluids over the fat tissue until blood and other contaminants are removed. The cleaned fat tissue then can be re-injected into the cannula using a delivery hose and other pressure source. In a preferred embodiment, the pump 86 and vacuum hose 88 could be used for both the liposuction and lipoinjection procedures. Cleaned fat tissue would travel down the length of the cannula and would be layered into bores in the patient's body parts made by the surgeon by deposition through the windows 74, 76, or 82. Thus, the use of a collection filter 90 in a liposuction/lipoinjection device provides the advantage of being able to more quickly wash and re-use excised fat tissue. Having the filter 90 in line with the vacuum hose allows the cleaning procedure to be performed immediately after liposuction. Alternatively, a wash line 94 could be connected to the filter 90 to allow cleaning to be performed during liposuction.

The fat collection filter 90 aspect of this invention can be used both with the liposuction/lipoinjection tool described above, and with conventional liposuction tools. All that is required is to provide a filter mechanism which allows isolation of fat tissue from other fluids during liposuction procedures. Prior art systems suffer from requiring a separate washing step to be performed on all of the collected tissue in the collection vessel 92 after the liposuction procedure is completed.

In a particular embodiment of this invention, the pump 86 or other vacuum pressure source could have controls 96 which allow the surgeon to adjust the vacuum pressure exerted at the cannula end. These controls 96 can take the form of dials, switches, buttons, or the like, and are designed to achieve vacuum pressures of varying strength. In most liposuction operations, a vacuum pressure ranging from 70-76 mm Hg is desired. However, greater vacuum pressures may be required if it is desired to use the liposuction tool of this invention in other applications. For example, this tool might also be used for removing bone chips in arthroscopic surgery, or removing cancerous lumps in biopsies, or in other applications. In addition to being able to select the type of cannula desired (e.g., selecting a cannula with large enough windows for cutting and removing cancerous tissue or bones), being able to adjust the vacuum pressure with controls 96 allows for the selective removal of different tissues. For example, at certain vacuum pressures only fat tissue will be aspirated into the windows of the cannula and removed from the patient's body, and surrounding muscle tissue will not be aspirated. However, if a cancerous lesion is desired to be removed, the surgeon would insert the cannula into the lesion and adjust the suction exerted by the pump 86 upward using controls 96.

FIG. 10 shows an embodiment of the invention wherein the cannula 98 includes an internal member 100 which is intended to assist in clearing the cannula 98 of fat tissue aspirated through window 99. Thus, in this embodiment, the internal member 100 is intended to prevent clogging during liposuction. The internal member 100 can take several different forms. In one embodiment, the internal member 100 delivers a gas (hydrofluorocarbons, oxygen, etc.) or fluid (water, saline, etc.) to the tip of the cannula 98, which, in addition to the vacuum pressure exerted by the pump or other suction device, is intended to help carry the fat tissue down the length of the cannula and into the vacuum hose. To assist in connecting a fluid or gas delivery mechanism to the internal member, the vacuum hose can be fabricated with an internal conduit which carries the fluid or gas to the internal member. In this way, a single connection of the vacuum hose will connect both the cannula and its internal member for both suction and fluid or gas delivery, respectively. In another embodiment, the internal member 100 heats the cannula in order to enhance skin tightening after surgery. For example, the internal member 100 could take the form of a wire or heating member that applies energy to the surface of the cannula.

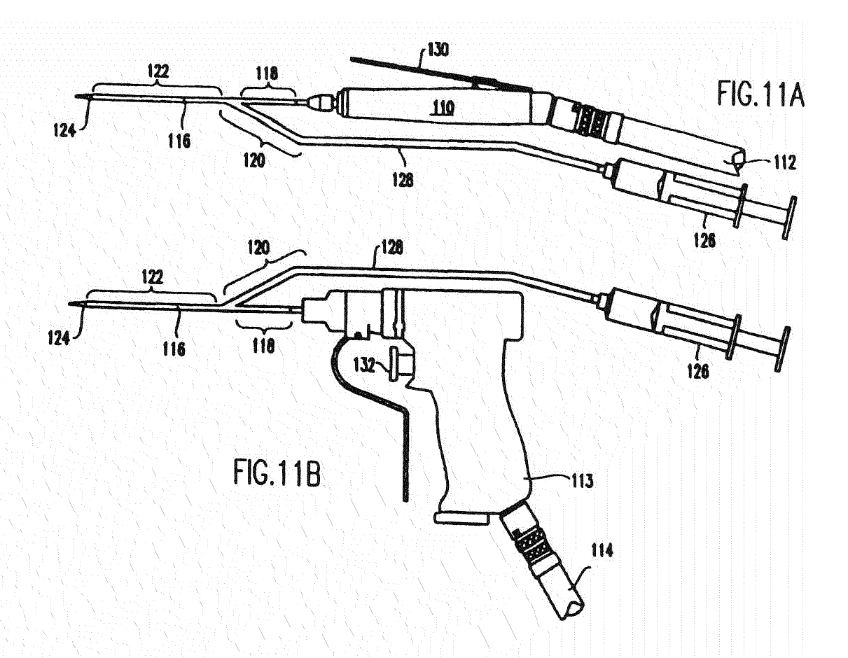

FIGS. 11A and 11B show alternative designs for the power-assisted liposuction handpiece of the present invention, each of which use a “Y” shaped cannula. FIG. 11A shows a “wand” style handpiece 110 connected to a pneumatic hose 112, while FIG. 11B shows a “pistol grip” style handpiece 113 connected to a pneumatic hose 114. A “Y” shaped cannula 116, having a drive arm region 118, a vacuum branch region 120, and an insertion tip region 122, is connected to the front portion of each handpiece 110 and 113. The tip 124 of the cannula 116 can be narrowed into a point or spatula shape as shown in FIGS. 8A and 8B, or can be blunt ended, bulled shaped, or assume any other configuration desired. Suction from source 126, which can be a syringe, pump, or other suitable device, is directed through vacuum hose 128 to the vacuum branch region 120 and into the insertion tip region 122. As discussed above, the cannula 116 is hollow and allows fat tissue to be withdrawn from the patient into the insertion tip region, through the vacuum branch region and into a collection vessel (not shown), under the pressure exerted by source 126. The drive arm branch 118 is connected to the handpiece 110 or 113 and, as described in detail above, the handpiece 110 or 113 reciprocates the cannula 116 back and forth. Lever 130 or trigger 132 can be used to vary the speed of reciprocation or simply to turn the reciprocating movement on and off. FIGS. 11A and 11B show that the same cannula 116 can be fitted onto different styles of handpieces, and it should be understood that the cannula 10 shown in FIGS. 11A and 11B can also be fitted onto different styles of handpieces in a similar fashion.

FIG. 12 shows a power-assisted laser liposuction handpiece with a laser (not shown) so that laser light is emitted from the tip 1050 of the cannula 1000. The cannula 1000 is safely retained on the handle 1020 by a connector 1024. The other end of the handpiece includes a connector 1018 to the power source (or compressed air source) and the vacuum hose 1026 used for liposuction, as described previously. The laser is preferably optimized to emulsify, liquefy or soften human or animal fatty tissue that is near the tip of the cannula 1000. An optical fiber projects the laser energy at the cutting windows from inside the end the cannula 1000 to the surrounding tissue so that it might be softened, emulsified or liquefied. As discussed above, the surrounding tissue is then suctioned into the cannula 1000 to remove it from the patient's body.

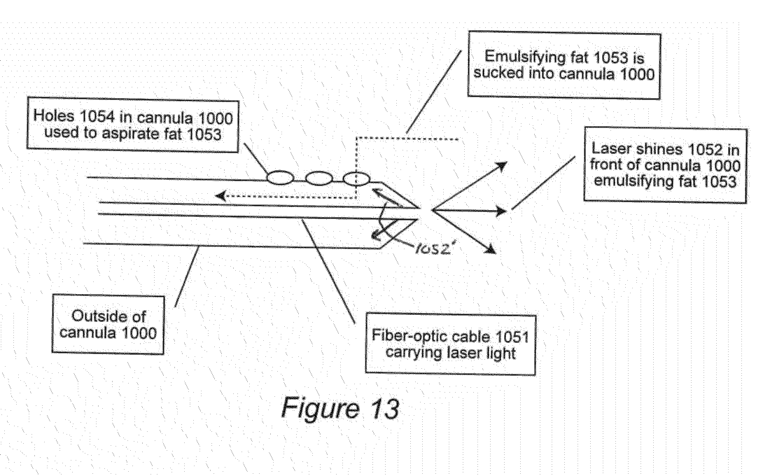

FIG. 13 illustrates the combined action of the laser and the suction function of an exemplary power assisted liposuction device. A laser light generated from a laser source is conducted via an optical fiber 1051 through the length of the cannula 1000 to its tip. Laser energy is emitted in front of the cannula as is indicated by arrows 1052 or emitted from a point inside the end of the cannula towards the cutting windows as indicated by arrows 1052′. As a result, fat 1053 of adipose tissues targeted by the surgeon are emulsified, softened or liquified immediately. In addition, the cannula 1000 of the present invention, on its outside wall and distal end, provides at least one or a series of holes 1054 which are used to aspirate the emulsified or liquefied fat 1053 into the cannula 1000 for storage or disposal. In a preferred embodiment, the holes 1054 can also be used to slice or cut fat for suctioning into the cannula as described above in conjunction with FIGS. 8A to 8D and elsewhere.

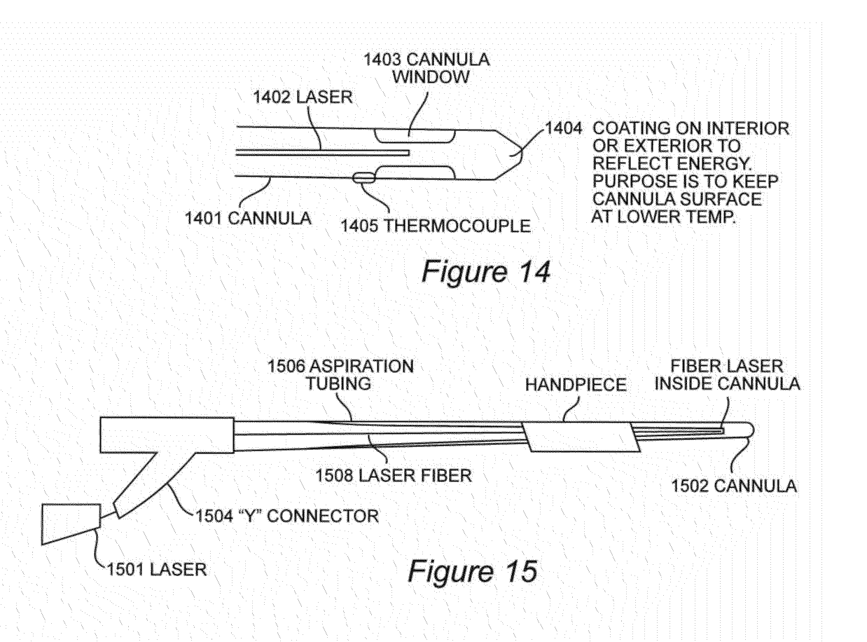

FIG. 14 shows a preferred embodiment of the invention where the cannula 1401 includes an optical fiber 1402 which carries radiant energy. e.g., laser energy, to a point near the top of the cannula 1401 just inside the cannula cutting windows 1403. In this embodiment, fat tissue located at the cutting windows 1403 which is either sliced or about to be sliced, is heated and melted or emulsified by the laser energy emanating from the end of the fiber 1402. This assists both in the ability to move the cannula 1401 back and forth through the tissue at the liposuction site, and in being able to suction the harvested tissue in the form of a liquid or softened tissue which will be less likely to clog the cannula or aspiration tube. The lasers which provide energy to the be delivered by the optical fiber 1402 in the invention could be Diode or Nd:YAG lasers in the infrared spectrum with current laser wavelengths to include those which have FDA clearance for laser lipolysis, which may include 924 nm, 980 nm, 1064 nm, 1320 nm, but could potentially range the spectrum from 500 nm to 2100 nm. There are some peaks where adipocytes preferentially absorb the infrared energy. The power range of the lasers could 5 watts to 50 watts, with 20 to 40 watts being the most common if larger laser fibers are used. In general, the power range is dependent on the internal surface area (and hence diameter) of the PAL cannula with larger power required for larger diameter cannulas compared with smaller diameter cannulas.

As noted in FIG. 14, the inside of the cannula 1401 or a portion of the inside of the cannula 1401 can be coated with a reflective coating 1404 (e.g., chrome, silver, gold, glass, etc.) which reflects the radiant energy from the end of the fiber 1402 or from an LED (not shown) at the end of a wire located where the fiber end 1402 is located. The reflection will keep the outer cannula surface at a lower temperature than if the reflective were not present. That is, without a reflective coating, the radiant energy from the fiber could heat the end of the cannula 1401 to a temperature less desirable for the liposuction procedure.

FIG. 14 also shows a thermocouple 1405 or other suitable temperature measuring device that can be used to monitor temperature changes for one or more of the cannula 1401/internal chamber and fat space/external chamber in the vicinity where the laser energy discharge occurs. Contemporaneous indwelling temperature monitoring in a power assisted liposuction tool will provide the surgeon with additional information to assist him or her in liposuction procedures.

FIG. 15 shows an embodiment for providing radiant energy from, for example, laser source 1501 to the cutting windows at the end of cannula 1502. Specifically, a “Y” connector 1504 can be fitted onto the aspiration tube 1506 (tubing 26 in FIG. 1) which permits optical fiber 1508 to pass into the aspiration tube 1506 without adversely affecting the suction capability. The optical fiber 1508 passes through the handpiece 1510 (preferably as shown in FIGS. 1A and 1B) and into the cannula 1502. As is best shown in FIG. 14, the optical fiber extends to the cutting windows of the cannula 1502 so that radiant energy is projected at tissue that is located at the cutting windows and which is either being harvested or about to be harvested. The configuration shown in FIG. 15 permits easy assembly of a power assisted liposuction handpiece with a confined laser beam according to a preferred embodiment of the present invention: however, it should be clear that other configurations for providing an energy source inside the cutting windows of the cannula can also be used in the practice of this invention.

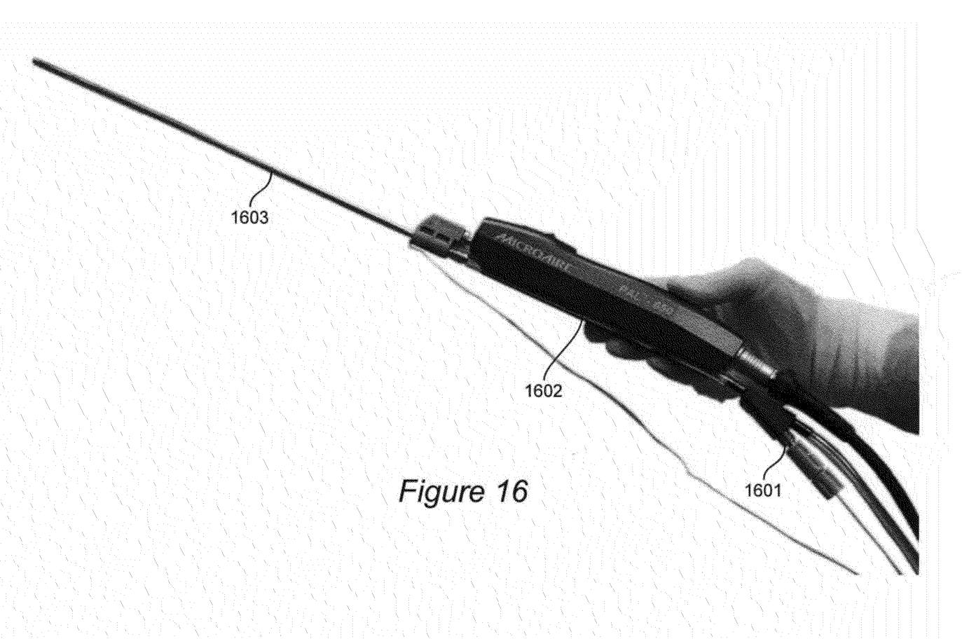

FIG. 16 shows an example of the power assisted liposuction tool with confined beam laser liposuction (CBLL) capability according to the present invention in the hand of a surgeon. For exemplary purposes, a distal portion of K-type thermocouple is attached to the Y adapter 1601 near the handpiece 1602. The reciprocating cannula 1603 is equipped with a CBLL as shown and discussed in FIGS. 14 and 15, such that radiant energy emanating near the cutting windows assists in fat collection as the cannula 1603 moves back and forth under the control of the surgeon.

While the invention has been described in terms of a single preferred embodiment, those skilled in the art will recognize that the invention can be practiced with modification within the spirit and scope of the appended claims.

Having thus described our invention, what we claim as new and desire to secure by Letters Patent is as follows:

1. A confined beam, power assisted liposuction handpiece, comprising:
a handle;
a cannula connected to said handle which is moveable in a reciprocating or vibrating movement with respect to said handle, said cannula having a hollow interior and at least one opening in a tip region which permits human or animal tissue to pass into an interior region of the cannula;
a suction device for applying suction to said interior region of the cannula to suction human or animal tissue which passes into the interior region of the cannula;
an optical fiber positioned within said interior region of the cannula and extending through the cannula and having an end located at said at least one opening; and
a laser optically coupled to said optical fiber so as to project laser energy through said optical fiber to be emitted at said end located at said at least one opening,
whereby energy emitted at said end of the optical fiber from inside said cannula in a vicinity of said at least one opening at said tip region of said cannula to surrounding human or animal tissue serves to emulsify, liquefy or soften said surrounding human or animal tissue.

2. The confined beam, power assisted liposuction handpiece of claim 1, wherein said cannula includes a reflective coating on at least a portion of its interior near said tip region for reflecting energy emitted by said optical fiber.

3. The confined beam, power assisted liposuction handpiece of claim 1, wherein said at least one opening in said cannula includes a plurality of openings.

4. The confined beam, power assisted liposuction handpiece of claim 1, wherein said at least one opening in said cannula includes an edge for cutting human or animal tissue adjacent said at least one opening.

5. The confined beam, power assisted liposuction handpiece of claim 1, wherein said suction device includes a vacuum hose, and wherein said optical fiber extends from said laser, through at least a portion of said vacuum hose and into said cannula.

6. The confined beam, power assisted liposuction handpiece of claim 5, further comprising a “Y” connector or “Y” section of the cannula having one leg of the “Y” connector or section fitted onto the vacuum hose and the other leg of the “Y” connector or section receiving the optical fiber to pass into the cannula without adversely affecting the suction capability.

7. The confined beam, power assisted liposuction handpiece of claim 5, wherein said vacuum hose is connected to a proximal end of said cannula.

8. The confined beam, power assisted liposuction handpiece of claim 1, further comprising a thermocouple positioned on said cannula for measuring one or more of temperature within said cannula and temperature outside said cannula.

9. The confined beam, power assisted liposuction handpiece of claim 1, further comprising:
a connector for securing the cannula to a reciprocating member and to a vacuum hose within the handle; and
a selectively actuatable member on the reciprocating member used to install and lock the connector to the reciprocating member, such that the cannula will be safely retained on the handle during liposuction.

10. A liposuction handpiece with a confined beam energy source, comprising:
a handle;
a cannula connected to said handle, said cannula having a forward end and a proximal end, said cannula having a window at its forward end for extracting fat; and
an energy source within said cannula which provides energy at said window for softening, melting or emulsifying fat at said window, said energy source comprising:
an optical fiber positioned within said interior region of the cannula and extending through the cannula and having an end located at said window; and
a laser optically coupled to said optical fiber so as to project laser energy through said optical fiber to be emitted at said end located at said window,
whereby energy emitted at said end of the optical fiber from inside said cannula in a vicinity of said window at said tip region of said cannula to surrounding human or animal tissue serves to emulsify, liquefy or soften said surrounding human or animal tissue.

11. The liposuction hand piece of claim 10, further comprising a means for reciprocating said cannula back and forth along in line with its longitudinal axis.

12. The liposuction handpiece of claim 10, further comprising a thermocouple positioned on said cannula for measuring one or more of temperature within said cannula and temperature outside said cannula.
